(19) United States
(12) Patent Application Publication
(10) Pub. No.: US 2016/0256452 A1
(43) Pub. Date: Sep. 8, 2016

(54) Composition Of Opioid Receptor Antagonists For Use In Treatment Of Constipation With Highly Increased Whole Gut Transit Time

(71) Applicant: Develco Pharma Schweiz AG, Pratteln (CH)

(72) Inventors: Helene Rey, Rosenau (FR); Marc Fischer, Hagenthal - Le-Bas (FR); Isabelle Golfier, Bantzenheim (FR); Christian Holl, Riehen (CH)

(21) Appl. No.: 14/828,034

(22) Filed: Aug. 17, 2015

(30) Foreign Application Priority Data
Mar. 6, 2015 (EP) 15170570.4

(51) Int. Cl. A61K 31/485

(52) U.S. Cl. CPC A61K 31/485

(57) ABSTRACT

The present invention relates to a composition comprising an opioid receptor antagonist in an extended release formulation for use in treating a patient with severe constipation characterised by a defined whole gut transit time.

### BACKGROUND

The present invention relates to a composition comprising an opioid receptor antagonist in an extended release formulation for use in treating a patient with severe constipation characterised by a highly increased whole gut transit time.

Constipation is a major symptom of bowel malfunction long-term consequences of constipation can be increased morbidity and mortality as well as deterioration of quality of life. Chronic constipation can result in hemorrhoid formation, rectal pain and burning, bowel obstruction and potential bowel rupture and death.

Traditional therapies for constipation include bulking agents, stool softeners, stimulant laxatives, and osmotic agents, but the effects of such therapies are nonspecific and are often generating diarrhea or cramps and some of these drugs cause severe side effects. Furthermore, these conventional measures are sometimes insufficient for some patients.

In some patients the constipation can be opioid induced. Opioid-induced constipation is predominantly due to the activity of gastrointestinal μ-opioid receptors. Selective inhibition of these peripheral receptors can relieve constipation without compromising centrally mediated effects of opioid analgesia or precipitating withdrawal.

Naloxone is a competitive antagonist of opioid receptors inside and outside of the central nervous system and is used in the treatment of opioid overdoses in that a naloxone solution is injected. When administered orally, it can reduce opioid-induced constipation in the gut. The advantage of naloxone is that it has a high first-pass metabolism. The laxative effect can therefore be achieved, without the narcotic analgesic effect of the opioid being significantly antagonised. In some patients, however, withdrawal symptoms or reduction of analgesia was observed upon naloxone administration.

Another way of preventing the antagonisation of the analgesic function of the opioid treatment is the use of opioid antagonists which cannot penetrate the blood-brain barrier, such as methylnaltrexone and alvimopan. Their inhibitory effect on μ-opioid receptors in the gastrointestinal tract appears to reverse opioid-induced gut hypomotility. Subcutaneous administration of methylnaltrexone (Relistor, Wyeth) is approved for the treatment of constipation in patients in palliative care that are under opioid treatment (EU SPC for Relistor, methylnaltrexone subcutaneous injection, Wyeth Europa Ltd., UK revised July 2008).

### BRIEF DESCRIPTION OF THE DRAWINGS

FIG. 1 shows the intestinal transit times in 15 subjects before and after pre-treatment with loperamide (LOP).

FIG. 2 shows the effect of subcutaneous (SC), immediate-release (IR) and extended-release (ER) administration of methylnaltrexone (MNTX) on the oro-cecal transit time (OCT) and the colon transit time (CU) in 15 subjects upon loperamide (LOP) treatment.

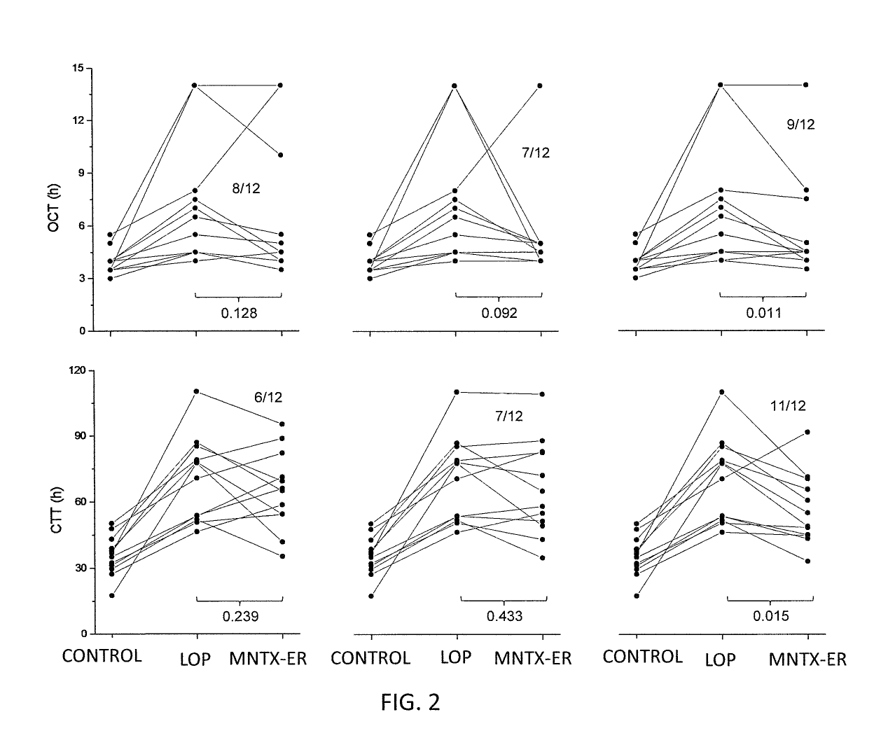

### DESCRIPTION OF THE INVENTION

The objective of the present invention is to provide a solid oral pharmaceutical composition comprising an opioid receptor antagonist, or a pharmaceutically acceptable salt or derivative thereof, as an active substance, wherein the composition releases the opioid receptor antagonist in an extended release formulation, for use in treating patient with severe constipation characterised by a highly increased whole gut transit time (WGT).

The present invention achieves those objectives by a composition comprising an opioid receptor antagonist, or any pharmaceutically acceptable salt or derivative thereof, for use in treating a patient with severe constipation, wherein
(i) the antagonist in the composition is prepared in an extended release formulation, and
(ii) the patient is characterized by severe constipation characterized by a whole gut transit time (WGT) of at least 65 h.

The feature “the antagonist in the composition is prepared in an extended release formulation” means that the composition comprising an opioid receptor antagonist releases the opioid receptor antagonist in a prolonged manner.

Preferably, the composition is a composition that is administered orally.

In a preferred embodiment, the severe constipation is characterized by a WGT of 65 to 180 h, preferably 65 to 150 h, more preferably of 70 to 140 h, even more preferably of 70 to 130 h, yet more preferably of 70 to 120 h, and most preferably of 70 to 110 h.

An amount of time defined by a number of hours is to be understood as the indicated amount of time in hours ±10 h, ±8 h, ±5 h, ±3 h, more preferably ±1 h, and even more preferably ±0.5 h, but most preferably, the stated time in hours.

In a further preferred embodiment, the severe constipation is not an opioid-induced constipation.

In an alternative preferred embodiment the severe constipation is an opioid-induced constipation.

Opioid-induced constipation can be caused by any opioid analgesic or opioid analgesic analogue, or by any of their salts or mixtures. Examples of such analgesics are the following: alfentanil, allylprodine, alphaprodine, anileridine, benzylmorphine, bezitramide, buprenorphine, butorphanol, clonitazene, codeine, besomorphine, dextromoramide, dezocine, diampromide, diamorphone, dihydrocodeine, dihydromorphine, dimenoxadol, Dimepheptanol, dimethylthiambutene, dioxaphetyl butyrate, dipipanone, eptazocine, ethoheptazine, ethylmethylthiambutene, ethylmorphine, etonitazene, fentanyl, heroin, hydrocodone, hydromorphone, hydroxypethidine, isomethadone, ketobemidone, levorphanol, levophenacylmorphane, lofentanil, meperidine, meptazinol, metazocine, methadone, metopone, morphine, myrophine, narceine, nicomorphine, norlevorphanol, normethadone, nalorphine, nalbuphene, normorphine, norpipanone, opium, oxycodone, oxymorphone, papaveretum, pentazocine, phenadoxone, phenomorphane, phenazocine, phenoperidine, piminodine, piritramide, propheptazine, promedol, properidine, propoxyphene, sufentanil, tilidine, and tramadol, wherein hydrocodone, morphine, hydromorphone, oxycodone, buprenorphine, codeine, fentanyl, levorphanol, meperidine, methadone, levomethadone, and dextromethadone are particularly preferred according to the invention.

The WGT time can be determined by any suitable method. However, in a preferred embodiment, the WGT time is determined using radio-opaque markers.

Alternatively the severe constipation can be characterized by the colon transit time. Colon transit time is defined as the Whole gut transit time minus the Oro-cecal transit time. Oro-cerecal transit time can be determined by any suitable method. Preferably, Oro-cecal transit time is determined by the sulfasalazine/sulfapyridine method (Gramatte, T. et al.; 1991, Int. J. Clin. Pharmacol. Ther. Toxicol.; 29(4), 147-150).

According to a further embodiment of the invention, the severe constipation is characterized by a colon transit time of at least 55 h. Preferably, the severe constipation is characterized by a colon transit time of 56 to 130 h, more preferably of 60 to 120 h, even more preferably of 65 to 110 h, yet more preferably of 70 to 105 h, and most preferably of 75 to 100 h.

Preferably, the opioid receptor antagonist is a μ-receptor antagonist.

The daily dosage of a μ-receptor antagonist in the composition can be expressed as naloxone-equivalent dosage. This relation is likely due to the fact that naloxone also belongs to the class of μ-receptor antagonists.

In one embodiment of the invention, the composition comprises the opioid receptor antagonist, or the pharmaceutically acceptable salt thereof, in a dose equivalent to 8 to 60 mg of naloxone, more preferably a dose equivalent to 10 to 56 mg of naloxone, even more preferably a dose equivalent to 12 to 48 mg of naloxone, and most preferably a dose equivalent to 12, 24, 36 or 48 mg of naloxone.

An amount of an opioid or of an opioid antagonist is to be understood as the indicated amount ±10%, ±8%, ±5%, ±3%, or more preferably ±1%.

In a preferred embodiment, the opioid receptor antagonist is naloxone, methylnaloxone, naloxonesulfonate, methylnaltrexone, naltrexone or any derivative, in particular esthers or sulfonates, or pharmaceutically acceptable salts thereof. More preferably, the opioid receptor antagonist is naloxone or methylnaltrexone, or any derivative or pharmaceutically acceptable salt thereof. In one preferred embodiment, the opioid receptor antagonist is naloxone hydrochloride. In an alternative preferred embodiment, the opioid receptor antagonist is methylnaltrexone, or even more preferably methylnaltrexone bromide.

N-methylnaltrexone bromide (methylnaltrexone) is a quaternary derivative of the pure opioid antagonist naltrexone that does not cross the human blood-brain barrier. Following subcutaneous administration, methylnaltrexone is rapidly absorbed, with peak concentrations achieved at approximately 0.5 hours after administration. The steady-state volume of distribution is 1.1 l/kg. The proportion of methylnaltrexone that binds to plasma proteins is 11-15% and its terminal half-life was found to be about 8 hours. Accumulation has not been observed with intravenous administration every 6 hours. Methylnaltrexone is primarily eliminated without being metabolised (85% of the administered dose). Approximately half of the dose is excreted in urine and less in faeces. A smaller part of the dose is metabolized to five distinct metabolites; N-demethylation to naltrexone is not a significantly used route.

The extended release form preferably comprises a defined and/or known release rate. The in vitro release rate is determined using the paddle stirrer apparatus (apparatus 2) with the paddle stirrer method according to Ph. Eur. (European Pharmacopoeia, 7th edition, 3rd supplement, 2.9.3 “Dissolution test for solid dosage forms“, pages 3797-3803) at 75 rpm in 500 ml 0.1 N hydrochloric acid at 37° C. The amount of released active substance is preferably determined by UV-detection at 220 nm.

Preferably, the release rate of the opioid receptor antagonist is measured using the paddle stirrer method according to Ph. Eur. at 75 rpm in 500 ml 0.1 N hydrochloric acid at 37° C., is 0% to 75% in 2 h, 3 % to 95 % in 4 h, 20 % to 100 % in 10 h, 30 % to 100 % in 16 h, 50 % to 100 % in 24 h, and of more than 80% in 36 h.

It was observed that the composition according to the invention, with its release profile, was suitable for an administration period of at least twelve-hours for the treatment of opioid-induced constipation. Accordingly it possesses a relatively high level of patient compliance.

In a preferred embodiment of the invention, the composition has an in vitro release rate of the active substance of 0% to 50% in 2 h, of 5 % to 95% in 4 h, of 20 % to 90 % in 10 h, of more than 70 % in 18 h, and of more than 80% in 24 h.

In a more preferred embodiment of the invention, the composition has an in vitro release rate of the active substance of 0% to 38 % in 2 h, of 5% to 55% in 4 h, and of 20 % to 75 % in 10 h.

According to another preferred embodiment of the invention, the composition has an in vitro release rate of the active substance of 0% to 50% in 1 h, of 10% to 95% in 4 h, of 35% to 100% in 8 h, of 55% to 100 % in 12 h, of 70% to 100 % in 16 h, and of more than 90% in 24 h.

According to another preferred embodiment of the invention, the composition has an in vitro release rate of the active substance of 0% to 30 % in 1 h, of 0 % to 40 % in 2 h, of 3% to 55% in 4 h, of 10% to 65% in 8 h, of 20% to 75% in 12 h, of 30% to 88% in 16 h, of 50% to 100% in 24 h, and of more than 80% in 36 h.

In a further preferred embodiment of the invention, the composition has an in vitro release rate of the active substance of 10% to 30% in 1 h, of 17 % to 37 % in 2 h, of 27 % to 47% in 4 h, of 40% to 60 % in 8 h, of 50 % to 70 % in 12 h, of 60 % to 80 % in 16 h, of 80 % to 100 % in 24 h.

In a particularly preferred embodiment of the invention, the composition releases the active substance independently of the ambient pH of the gastrointestinal tract. This ensures that the entire gastrointestinal tract can be evenly and continuously supplied with the opioid receptor antagonist, or an acceptable salt thereof. A further optimisation of the treatment is thereby achieved. The pH-independent release of the active substance from the composition of the invention can be achieved through the choice of suitable pharmaceutical excipients that will be known to the person skilled in the art. Local pH values in the gastrointestinal tract are from about 1.2 (in the stomach), to about 6.8 in the colon.

The release of the active substance from the composition of the invention that is independent from the pH of the gastrointestinal tract is preferably understood to mean that the similarity factor f2 between a first in vitro release at a pH of 1.2 to 6.8 and a second in vitro release at any other pH of 1.2 to 6.8 is larger or equal to 50.

The similarity factor f2 is determined according to SHAH V. P., TSONG Y., SATHE P., & LIU J. P. (1998), “In vitro dissolution profile comparison-statistics and analysis of the similarity factor, f2”, Pharmaceutical Research, 15, 889-896. Specifically, the similarity factor f2 is calculated by the following formula:

$${f}_{2} = 50 * {log}_{10} ( {[ 1 + \frac{1}{n} {\sum }_{t = 1}^{n} {( {R}_{t} - {T}_{t} )}^{2} ]}^{0.5} * 100 )$$

In this equation, Rt and Tt represent the released quantities of active substance at time point t at the first and second pH. n is the number of time points. The f2 factor is determined under the following conditions: a) the minimal number of time points for one release is 3 (time point 0 is excluded); b) the time points for the first and the second pH should be equal; c) for each time point, and for each pH, the released quantity is indicated as the mean value of 12 measurements; d) no more than one mean value measured above a release of 85% can be taken into account for the calculation; e) the relative standard deviation or coefficient of variation of the release at a given pH should be smaller than 20% for the first time point and smaller than 10% for the second, and every subsequent time point.

The composition according to the invention may comprise a matrix, which releases an opioid receptor antagonist such as naloxone, or a pharmaceutically acceptable salt thereof or any derivative, in a prolonged manner. The matrix according to the invention is preferably a so-called scaffold matrix, which can be swelling or non-swelling, or can be a so-called eroding matrix. The matrix can also have properties of both scaffold and eroding matrixes.

In the case of a scaffold matrix, the active substance is incorporated into the matrix structure. The active substance is gradually dissolved by the digestive juices from the loaded scaffold matrix during the transport through the gastrointestinal tract. At the end of the process, the matrix scaffold is excreted in more or less unchanged form, or in a swollen form. In contrast, with an eroding matrix, the matrix is degraded, or eroded, which leads to active substance particles being exposed at the surface, and dissolved. The release rate therefore depends on the matrix degradation or erosion rate.

For the purpose of forming a largely stable scaffold matrix with an appropriate active substance release rate, a further preferred embodiment of the invention is a composition with a matrix that comprises one or several water-insoluble matrix-forming agents. Another embodiment of the invention is a composition with a matrix that comprises one or several water-soluble matrix-forming agents.

According to a further preferred embodiment of the invention, the matrix of the composition is water-insoluble. In an alternative embodiment of the invention, the matrix of the composition is water-soluble.

In another preferred embodiment of the invention, the matrix of the composition comprises one or several matrix-forming agents selected from the group consisting of cellulose esters, polyethylene oxide, polyvinylpyrrolidone/polyvinyl acetate mixtures, methacrylate-acrylate copolymers, waxes, fats such as glycerol esters, and fatty alcohols. The substance classes mentioned here are particularly suitable as matrix-forming agents for the composition of the invention. However, particularly preferred is the use of a mixture of polyvinyl acetate and polyvinylpyrrolidone, and/or a glycerol dibehenic acid ester as matrix-forming agent.

In a further preferred embodiment of the invention, the composition is free of film-coated, opioid receptor antagonist-containing particles, wherein the coating causes the prolonged release of the opioid receptor antagonist.

According to a further preferred embodiment of the invention, the composition can be formed by direct compression, since this is particularly inexpensive.

According to another preferred embodiment of the invention, the composition is in the form of a tablet, capsule, granule, a micro tablet, extruded particles or granules compressed into a tablet.

In a further preferred embodiment of the invention, the composition is designed as a once-a-day formulation, or a twice-a-day formulation.

In another embodiment, one or several further active substances can be present in the composition, in addition to the opioid receptor antagonist.

Regarding the composition which is particularly suited for a twice-a-day administration, the present invention further relates to a solid oral pharmaceutical composition comprising an opioid receptor antagonist, or a pharmaceutically acceptable salt thereof, as an active substance, wherein the composition releases the active substance in a prolonged manner, and the in vitro release rate of the active substance, measured using the paddle stirrer method according to Ph. Eur. at 75 rpm in 500 ml 0.1 N hydrochloric acid at 37° C., is of 5% to 50% in 1 h, of 10 % to 75% in 2 h, of 20 % to 95% in 4 h, of 40% to 100% in 8 h, of more than 50% in 12 h, of more than 70% in 18 h, and of more than 80% in 24 h.

Regarding the composition which is particularly suited for a twice-a-day administration, the present invention further relates to a solid oral pharmaceutical composition comprising an opioid receptor antagonist, or a pharmaceutically acceptable salt thereof or any derivative, as an active substance, wherein the composition releases the active substance in a prolonged manner, and the in vitro release rate of the active substance, measured using the paddle stirrer method according to Ph. Eur. at 75 rpm in 500 ml 0.1 N hydrochloric acid at 37° C., is of 20% to 50% in 1 h, of 40% to 75% in 2 h, of 60 % to 95 % in 4 h, of 80 % to 100 % in 8 h, and of 90 % to 100 % in 12 h.

Regarding the composition which is particularly suited for a once-a-day administration, the present invention further relates to a solid oral pharmaceutical composition comprising an opioid receptor antagonist, or a pharmaceutically acceptable salt thereof or any derivative, as an active substance, wherein the composition releases the active substance in a prolonged manner, and the in vitro release rate of the active substance, measured using the paddle stirrer method according to Ph. Eur. at 75 rpm in 500 ml 0.1 N hydrochloric acid at 37° C., is of 0% to 50% in 1 h, of 0% to 75% in 2 h, of 10 % to 95 % in 4h, of 35 % to 100 % in 8 h, of 55 % to 100 % in 12 h, of 70 % to 100 % in 16 h and of more than 90% in 24h.

Regarding the composition which is particularly suited for a once-a-day administration, the present invention further relates to a solid oral pharmaceutical composition comprising an opioid receptor antagonist, or a pharmaceutically acceptable salt thereof or any derivative, as an active substance, wherein the composition releases the active substance in a prolonged manner, and the in vitro release rate of the active substance, measured using the paddle stirrer method according to Ph. Eur. at 75 rpm in 500 ml 0.1 N hydrochloric acid at 37° C., is of 0% to 30% in 1 h, of 0% to 40% in 2 h, of 3% to 55% in 4 h, of 10 % to 60 % in 8 h, of 20 % to 75% in 12 h, of 30 % to 88% in 16 h, of 50 % to 100% in 24 h, and of more than 80% in 36h.

Regarding the composition which is particularly suited for a once-a-day administration, the present invention further relates to a solid oral pharmaceutical composition comprising an opioid receptor antagonist, or a pharmaceutically acceptable salt thereof, as an active substance, wherein the composition releases the active substance in a prolonged manner, and the in vitro release rate of the active substance, measured using the paddle stirrer method according to Ph. Eur. at 75 rpm in 500 ml 0.1 N hydrochloric acid at 37° C., is of 10 % to 30 % in 1 h, of 17% to 37 % in 2 h, of 27% to 47% in 4 h, of 40 % to 60 % in 8 h, of 50 % to 70 % in 12 h, of 60 % to 80 % in 16 h, and of 80 % to 100 % in 24 h.

In accordance with good patient compliance, a further preferred embodiment of the invention is a composition, wherein the composition is preferably a tablet or a capsule, which has an in vitro release rate of the active substance, measured using the paddle stirrer method according to Ph. Eur. at 75 rpm in 500 ml 0.1 N hydrochloric acid at 37° C., of 0% to 75% in 2 h, of 3% to 95% in 4 h, of 20% to 100 % in 10 h, of 30 % to 100 % in 16 h, of 50 % to 100 % in 24 h, and of more than 80 % in 36 h.

The release rate is, in accordance with the invention, controlled by adjusting the mass ratio of opioid receptor antagonist to matrix-forming agent. In a preferred embodiment, the mass ratio of opioid receptor antagonist to matrix-forming agent is 1:1, more preferably 1:2, more preferably 1:5, more preferably 1:10, more preferably 1:20, even more preferably 1:50, yet more preferably 1:75 and most preferably 1:100.

The composition of the invention is characterised in that through the prolonged release the concentration of the opioid receptor antagonist in the plasma is low. Its maximum plasma concentration (Cₘₐₓ) is about 20× lower during the active course compared to a composition without prolonged release, and about 100× lower compared with an intravenously administered composition.

The inhibition of the receptors over the active course is advantageous. In addition to providing the constipation prevention effect of the opioid receptor antagonist, the low bioavailability in the system also ensures a reduced likelihood and/or severity of the side effects.

Since the naloxone inhibitory concentrations (IC₅₀) for opioid receptors (μ, δ and κ) are known, the assessment of the risk factor of a tablet can be calculated with the ratio IC₅₀/Cₘₐₓ. With the IC₅₀ of μ receptor, the value of IC₅₀/Cₘₐₓ for a tablet according to the invention with 48 mg of naloxone is 54. In general, the higher the value of IC₅₀/Cₘₐₓ, the lower the risk factor of the tablet according to the invention. Hereafter all values relating to the IC₅₀ are for the Li receptor.

In a preferred embodiment, the composition has an IC₅₀/Cₘₐₓ value of at least 30. In a more preferred embodiment, the composition has an IC₅₀/Cₘₐₓ value of at least 35. In an even more preferred embodiment, the composition has an IC₅₀/Cₘₐₓ value of at least 40. In the most preferred embodiment, the composition has an IC₅₀/Cₘₐₓ value of at least 50.

In a further embodiment, the composition additionally comprises at least one stabilizer, which protects the active substance. In a preferred embodiment, the at least one stabilizer is selected from the list comprising sulphur dioxide, sodium sulphite, sodium bisulphite, ascorbic acid and its derivatives and tocopherol, as well as its water- and fat-soluble derivatives, such as, for example, tocopherol acetate, sulphites, bisulphites and hydrogen sulphites of alkali, alkaline earth metals or other metals, paraben, BHA, BHT, gallates, as well as lower fatty acids, fruit acids, phosphoric acids, sorbic and benzoic acids as well as their salts, esters, derivatives and isomeric compounds, ascorbyl palmitate, lecithins, mono- and polyhydroxylated benzene derivatives, ethylenediaminetetraacetic acid and salts thereof, citraconic acid, cysteine, L-cysteine, conidendrin, diethyl carbonate, methylenedioxyphenols, cephalin, β,β′-dithiopropionic acid, biphenyl and other phenyl derivatives.

In a further embodiment, the composition additionally comprises at least one stabilizer, which protects the matrix. In a preferred embodiment, the at least one stabilizer is selected from the list comprisingsulphur dioxide, sodium sulphite, sodium bisulphite, ascorbic acid and its derivatives and tocopherol, as well as its water- and fat-soluble derivatives, such as, for example, tocopherol acetate, sulphites, bisulphites and hydrogen sulphites of alkali, alkaline earth metals and other metals, paraben, BHA, BHT, gallates as well as lower fatty acids, fruit acids, phosphoric acids, sorbic and benzoic acids and their salts, esters, derivatives and isomeric compounds, ascorbyl palmitate, lecithins, mono- and polyhydroxylated benzene derivatives, ethylenediaminetetraacetic acid and their salts, citraconic acid, cysteine, L-cysteine, conidendrin, diethyl carbonate, methylenedioxyphenole, cephalin, β,β′-dithiopropionic acid, biphenyl and other phenyl derivatives.

In a further embodiment, the composition comprises at least one additive, wherein the additive is an emetic or a pungent agent drug. In a preferred embodiment, the composition comprises an additive, wherein this additive is a pungent agent, selected from the group comprising Allii sativi bulb, Asari rhizome cum herba, Calami rhizoma, capsici fructus (capsicum} capsici fructus acer (cayenne pepper), Rhizoma Curcumae Longae, Curcumae xanthorrhizae rhizoma, Galangae rhizoma, Semen Myristicae, Piperis nigri fructus (pepper), Sinapis albae (Erucae) Semen, Sinapis nigrae semen, Zedoariae rhizoma and Zingiberis rhizoma, preferably from the group consisting of capsici fructus (capsicum), capsici fructus acer (cayenne pepper) and Piperis nigri fructus (pepper).

In a preferred embodiment, the composition comprises at least one additive, wherein this additive is an emetic. In a preferred embodiment, the emetic is based on one or several substances from radix ipecacuanha (ipecac). In a preferred embodiment, the emetic is based on the substance emetine, in an alternative embodiment, the emetic is apomorphine.

In a further embodiment, the composition comprises a dye. In a preferred embodiment, the dye is selected from a group comprising red iron oxide, black iron oxide and indigo carmine.

In a further embodiment, the composition additionally comprises at least one non-steroid antirheumatic or an antihistamine.

In an alternative embodiment, the composition additionally comprises at least one water-soluble lubricant. In a preferred embodiment, the composition comprises at least one water-soluble lubricant selected from the group comprising adipic acid, fumaric acid, sodium benzoate and macrogol.

Disclosed are methods comprising administering a composition comprising an opioid receptor antagonist to a subject having severe constipation characterized by a whole gut transit time (WGT) of at least 65 h. In some instances, the opioid receptor antagonist can be a μ-receptor antagonist. For example, the μ-receptor antagonist can be, but is not limited to, naloxone, naloxonesulfate, methylnaloxone, naltrexone, or a pharmaceutically acceptable salt thereof.

Disclosed are methods comprising administering a composition comprising an opioid receptor antagonist to a subject having severe constipation characterized by a WGT of at least 65 h, wherein the severe constipation is not an opioid-induced constipation.

Disclosed are methods comprising administering a composition comprising an opioid receptor antagonist to a subject having severe constipation characterized by a WGT of at least 65 h, wherein the whole gut transit time is determined using radio-opaque markers. WGT can be determined using any method well known in the art.

Disclosed are methods comprising administering a composition comprising an opioid receptor antagonist to a subject having severe constipation characterized by a WGT of at least 65 h, wherein the patient is characterized by a whole gut transit time of at least 65 to 100 h.

Disclosed are methods comprising administering a composition comprising an opioid receptor antagonist to a subject having severe constipation characterized by a WGT of at least 65 h, wherein the composition releases the opioid receptor antagonist independently of the ambient pH of the gastrointestinal tract.

Disclosed are methods comprising administering a composition comprising an opioid receptor antagonist to a subject having severe constipation characterized by a WGT of at least 65 h, wherein the composition is designed as a once-a-day formulation. In some instances, the composition can be a twice-a-day formulation. For example, the extended release formulation can have a 12-hour or a 24-hour release rate.

Disclosed are methods comprising administering a composition comprising an opioid receptor antagonist to a subject having severe constipation characterized by a WGT of at least 65 h, wherein the WGT comprises a colon transit time of 55 to 90 h. In some instances, the WGT is 65 to 180 h.

Disclosed are methods comprising administering a composition comprising an opioid receptor antagonist to a subject having severe constipation characterized by a WGT of at least 65 h, wherein the composition can further comprises a matrix.

Disclosed are methods comprising administering a composition comprising an opioid receptor antagonist to a subject having severe constipation characterized by a WGT of at least 65 h, wherein the composition is formulated as a capsule.

Disclosed are methods comprising administering a composition comprising an opioid receptor antagonist to a subject having severe constipation characterized by a WGT of at least 65 h, wherein the opioid receptor antagonist is not naloxone.

Disclosed are methods comprising administering a composition comprising an opioid receptor antagonist to a subject having severe constipation characterized by a WGT of at least 65 h, wherein the opioid receptor antagonist is not naloxone, and wherein the composition further comprises an opioid. Disclosed are methods comprising administering a composition comprising an opioid receptor antagonist to a subject having severe constipation characterized by a WGT of at least 65 h, wherein the severe constipation is an opioid-induced constipation. In some instances, the opioid-induced constipation is not caused by oxycodone.

### EXAMPLES

### Effect of the Opiod Antagonist Administration of Whole Gut Transmit Time

### Subjects

The clinical study was performed in 15 German white subjects (11 males, 4 females, age 20-34 years, body mass index 20.3-27.0 kg/ m²). All subjects were in good health as confirmed by medical histories, physical examination, routine clinical-chemical and hematologic screenings and 12-lead ECG. Three female subjects took hormonal contraceptives regularly and one subject took 300 mg ibuprofen to treat headache 4 days before the administration of study medication. Three subjects were smokers of less than 10 cigarettes per day. 14 subjects drank alcohol occasionally. All were negative in the pre-study screenings for alcohol, drugs, hepatitis-B-virus (HBV) and hepatitis-C-virus (HCV) and human immunodeficiency virus (HIV). Before administration of the study medication, female subjects were screened to be not pregnant and to apply a safe method of contraception. During the entire study period, the subjects avoided strenuous physical activities and did not eat or drink food or beverages containing grapefruit, poppy seeds or alcohol. All participants provided informed written consent before inclusion into the study. The study was approved by the Independent Ethics Committee of the University Medicine Greifswald and by the German Federal Institute for Drugs and Medical Devices (BfArM) and was registered by EudraCT (2009-014357-32) and ClinicalTrials.gov (NCT01596777).

### Study Protocol

Loperamide is an agonist of μ-opioid receptors in the myenteric plexus without central nervous opioid effects which is known to delay OCT of radiolabeled lactulose that can be reversed by the opioid antagonist naloxone.

Pharmacokinetics of methylnaltrexone and its effects on loperamid-induced experimental constipation were evaluated in a randomized, controlled, five-period, cross-over study with 7 days wash-out between the study periods. Experimental constipation was induced by 4 mg loperamide hydrochloride (Loperamide-ratiopharm®, ratiopharm, Germany) dissolved in 200 ml apple juice which was administered 24, 12 and 1 h before and 12 h after administration of methylnaltrexone placebo capsules (LOP), MNTX-SC (12 mg methylnaltrexone bromide, Relistor®, Wyeth, Berkshire, UK), MNTX-IR (500 mg methylnaltrexone bromide capsules, Develco Pharma Schweiz AG, Switzerland) and MNTX-ER (500 mg methylnaltrexone bromide capsules, Develco Pharma Schweiz AG, Switzerland), respectively. The time of methylnaltrexone administration was defined to be time zero. For control of intestinal transit times in absence of loperamide and methylnaltrexone, 200 ml of pure apple juice and a methylnaltrexone placebo capsule were given at the respective times (Control). Methylnaltrexone placebo capsules were identical in color and shape to MNTX-IR and MNTX-ER which all were swallowed using 200 ml table water; i.e., the clinical study has been performed double-blinded with exception of the MNTX-SC study period. Oro-cecal transit time (OCT) was assessed using the sulfasalazine/sulfapyridine method. For that, 500 mg immediate-release sulfasalazine (Azulfidine®, Pharmacia, Germany) were swallowed with 200 ml table water 2 h after administration of the respective methylnaltrexone study medication to avoid competitive interactions during absorption. The time of first appearance of sulfapyridine in serum was defined to be the OCT (cut-off: 100 μg/ml serum). Whole gut transit time (WGT) was evaluated using a radio-opaque marker method. For that, Colon Transit' capsules (Medical Instruments Corporation GmbH, Herford, Germany) were swallowed 24, 12 and 1 h before time zero using the 200 ml apple juice for loperamide dosing. Each capsule contains 10 radio-opaque markers of identical shape (e.g. triangles). A Colon Transit™ batch consists of capsules with markers of different shape (e.g. triangles, circles, rings etc.). Our subjects were willing and compliant for sampling stool in single portions immediately after feeling urge without suppressing defecation using tightly closable containers. Then, the number of radio-opaque markers with identical shape was counted in the stool portions after X-ray imaging (Philips Optimus, Philips Healthcare, Hamburg, Germany) using the Agfa PACS Workstation Impact-Version 5.2 (Agfa-Healthcare, Cologne, Germany). From the time of administration of the 3×10 radio-opaque markers (3 Colon Transit™ capsules) and the time of their appearance in different stool portions could be reliably concluded on WGT as described below. Colon transit time (CTT) was derived from the difference of WGT and OCT.

For supervised pretreatment with loperamide and Colon Transit™ (mouth checking) and for providing stool samples, the subjects visited the study unit. For pharmacokinetic evaluation and assessment of OCT using the sulfasalazine/sulfapyridine method, the subjects were admitted to the study unit in the evening before and remained there until last blood sampling. After overnight fasting, Colon Transit™, loperamide, the methylnaltrexone study medication or placebo and sulfasalazine were administered as described above. Bed rest lasted from 1.5 h before to 4.5 h after methylnaltrexone administration. Standard meals were served 5, 8 and 13 h after methylnaltrexone administration. The subjects had to eat and drink the same individual amount of food and table water on all study days. To evaluate methylnaltrexone pharmacokinetics and OCT with the sulfasalazine/sulfapyridine method, venous blood was sampled via an indwelling cannula placed in a forearm vein or by individual vein punctures 1.5 h before and 0.25, 0.5, 0.75, 1, 1.5, 2, 2.5, 3.5, 4, 4.5, 5, 5.5, 6, 6.5, 7, 7.5, 8, 8.5, 9, 9.5, 10, 12, 16, 24 h after administration of the study medication (Smonovette®, Sarstedt, Nürnbrecht, Germany). Urine was collected at 24 h-intervals for 3 days and stool was collected as described above. Serum and urine aliquots were stored at least at −20° C. until quantitative analysis of methylnaltrexone and sulfapyridine, respectively.

### Drug Analysis

Methylnaltrexone in human serum and urine and sulfapyridine in serum were assayed using a modified liquid chromatopraphy-tandem mass spectrometry (LC-MS/MS) method after protein precipitation of the samples using perchloric acid and with naltrexone as internal standard. [20, 21] The LC-MS/MS system consisted of the Agilent 1100 series HPLC system (Agilent Technologies, Waldbronn, Germany) equipped with the analytical column XTerra MS (2.1×100 mm, 3 μm, Waters, Milford, USA) and coupled with the tandem mass spectrometer API 4000 (AB Sciex, Darmstadt, Germany). The method was validated for simultaneous determination of methylnaltrexone and sulfapyridine in serum for the ranges of 0.5 to 250 ng/ml and 0.5 to 500 ng/ml, respectively. In urine, the calibration range for methylnaltrexone was 0.005-1.0 μg/ml.

The chromatograms were evaluated using the Analyst 1.4 software (AB Sciex, Darmstadt, Germany) with the internal standard method using peak-area-ratios for calculation. Between-day and within-day accuracy for the assays of methylnaltrexone in serum and urine and for sulfapyridine in serum was between +8.4% and −6.9% of the nominal values. Precision accounted for ±13.1% of the respective means.

### Biometrical Evaluation

Pharmacokinetics: Maximum serum concentrations (Cmax) and the time to reach Cmax (Tmax) were obtained from the concentration-time curves. The area under the concentrations-time curve was calculated using the trapezoidal formula with the measured data points from the time of administration until the last quantifiable concentration (AUC₀₋ₜ) and extrapolated to infinity (AUC_{0-∞}). Terminal elimination half-life (T_{1/2}) was estimated by log-linear regression analysis of the terminal slope. Renal clearance (CLR) was derived from cumulative urinary excretion (Aₑ) of methylnaltexone over AUC_{0-∞} after subcutaneous and oral administrations.

Intestinal transit times: Oro-cecal transit time (OCT) was defined as the time span between oral administration of sulfasalazine (500 mg immediate-release tablet) and the appearance of its metabolite sulfapyridine in serum (cut-off=100 μg/ml) which is generated in the cecum by bacterial azo-reduction. Whole-gut transit time (WGT) was assessed by counting the radio-opaque markers with different shapes (Colon Transit™) in the feces according to formula (1) :

$$WGT = \frac{{δ}_{1} + {δ}_{2} + {δ}_{3}}{3} \qquad ( 1 )$$

with δ₁₋₃ as derived by formula (2):

$${δ}_{t} = \frac{{\sum }_{n = 1}^{n} {x}_{i} \cdot {t}_{i}}{{\sum }_{n = 1}^{n} {x}_{i}} \qquad ( 2 )$$

δₜ =mouth to stool transit time of markers type 1, 2, 3
xᵢ=number of markers type 1, 2, 3 counted at time ti
n=total number of stools

The CTT was evaluated from the difference of WGT and OCT.

Sample size considerations: A post-hoc sample size evaluation using the data of the study (Table 2) and assuming an alpha-error of 0.05 and a power of 80% has shown that reductions of LOP-delayed OCT, CTT and WGT by 24%, 20% and 18%, respectively, can be confirmed with N=12 subjects.

Statistics: For all samples, arithmetic means (M)±SD are given. Sample differences were evaluated using the non-parametric Wilcoxon test as appropriate.

All statistical and pharmacokinetic calculations were performed with the SPSS version 12.0 (SPSS Inc., Chicago, U.S.A) and the SAS statistical package (SAS 8.02, SAS Institute Inc., Carry, U.S.A).

### Results

MNTX-SC was rapidly absorbed into the blood and reached maximum concentrations between 94.7 and 309 ng/ml after 0.25 to 0.75 hours. The volume of distribution (Vss) ranged between 107 and 712 L and the terminal elimination half-life between 3.4 and 26 hours. 22-51% of the dose was excreted unchanged into the urine. Renal clearance was between 233-707 ml/min and accounted for 40-51% of the total body clearance. As renal clearance exceeded 2-6 times the normal values of the glomerular filtration rate in healthy subjects (about 120 mg/min), a substantial part of the parent drug must have been eliminated by tubular secretion (Table 1).

TABLE 1
Pharmacokinetic characteristics (means ± SD) of methylnaltrexone
after subcutaneous injection of 12 mg (MNTX-SC) and single oral
administration of 500 mg in immediate-release capsules (MNTX-IR)
and extended-release capsules (MNTX-ER) in 15 healthy subjects.
T½ after administration of MNTX-ER was not assessed
because of the too low number of data points along the terminal
elimination slope that were above the limit of quantification.

| | | MNTX-SC | MNTX-IR | MNTX-ER |
|---|---|---|---|---|
| AUC_{0-∞} | (ng × h/mL) | 195 ± 53.2 | 252 ± 110 | 47.1 ± 34.7* |
| Cₘₐₓ | (ng/mL) | 143 ± 54.4 | 45.7 ± 25.1 | 4.63 ± 4.16* |
| Tₘₐₓ | (h) | 0.33 ± 0.15 | 2.57 ± 1.92 | 3.74 ± 1.38 |
| F | (%) | — | 3.11 ± 1.16 | 0.54 ± 0.31 |
| T_{1/2} | (h) | 9.23 ± 5.77 | 9.99 ± 7.90 | n.a. |
| CL_{R} | (mL/min) | 399 ± 132 | 404 ± 140 | 554 ± 415 |
| Aeᵤᵣᵢₙₑ | (mg) | 4.45 ± 1.05 | 5.72 ± 2.76 | 1.01 ± 0.42* |

*p < 0.05, compared to MNTX-IR (Wilcoxon test)

The absorption of MNTX-IR and MNTX-ER from the gastrointestinal tract was significantly delayed and rather incomplete. MNTX-ER was absorbed with significantly lower rate and was less bioavailable than MNTX-IR. Relative bioavailability of MNTX-IR and MNTX-ER was only 1.53-5.49% and 0.11-1.24% %, respectively, compared to MNTX-SC. Therefore, systemic exposure (AUC) of MNTX-IR and MNTX-ER was substantially smaller compared to MNTX-SC despite the 42-fold difference in dose (500 mg vs. 12 mg). Systemic elimination of MNTX-IR was not different from MNTX-SC as confirmed by similar terminal elimination halflives and renal clearances. For MNTX-ER, terminal half-life was not assessed because of low bioavailability and impossibility to assess the terminal slope correctly with an adequate number of data points.

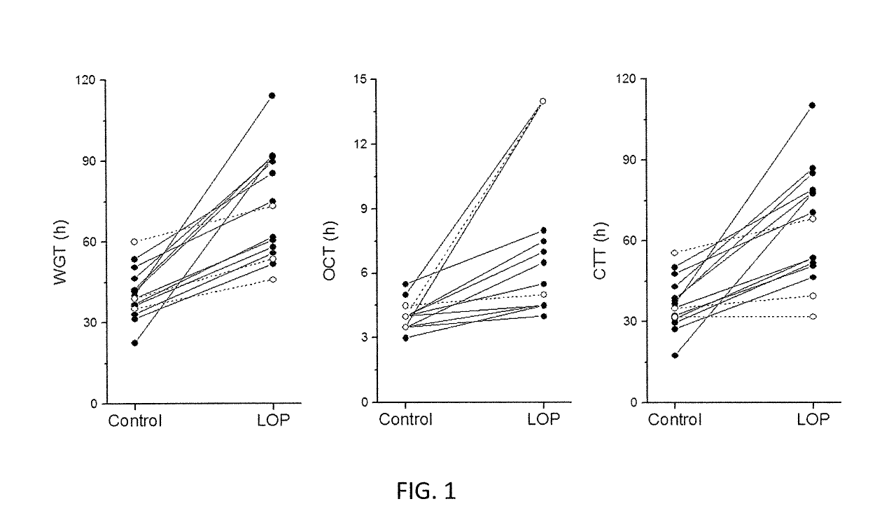

Intestinal transit times in healthy subjects without pretreatment with loperamide and methylnaltrexone (Control) were as follows; WGT ranged between 22.5 and 60.1 h (40.6±9.4 h) whereby OCT accounted for 2.8 and 5.3 h (3.9±0.6 h) and CTT for 17.7-55.6 h (36.7±9.6 h). After pre-treatment with loperamide (LOP), intestinal transit times increased in all 15 subjects (FIG. 1). WGT elevated about 1.8-fold to values of 45.9-114 h (73.4±19.9 h) with OCT of 4.0-14.0 h (7.4±3.7 h) and CCT of 33.9-110 h (66.0±20.9 h).

The effects of methylnaltrexone on loperamide-induced constipation was evaluated only in 12 subjects which responded with prolongation of the WGT by >20.5 h (>25% quartile of all WGT differences between Control versus LOP in 15 subjects). In this group of “LOP-responders”, the WGT extended by 20.6-74.1 h (37.8±19.1 h) with 0.50-10.5 h (3.08±3.30 h) being caused by prolongation of the OCT and 18.3-73.6 h (34.8±18.3 h) by CTT. MNTXSC was without significant effect on OCT and CTT. However, OCT was reduced almost to baseline in 8 of our 12 subjects. MNTX-IR also tended to influence OCT (p=0.092) which was reduced to baseline levels in 7 of our 12 subjects. MNTX-ER significantly antagonized loperamide effects on OCT and CTT but, however, not to baseline levels; the transit along the small and large intestine was still significantly prolonged after single dose co-medication of MNTX-ER (Table 2).

TABLE 2
Means ± SD of whole-gut transit time (WGT), oro-cecal transit time (OCT) and
colon transit time (CTT) before (Control) and after loperamide induced constipation (LOP)
and after co-medication of methylnaltrexone by subcutaneous injection of 12 mg (MNTX-
SC) and single oral administration of 500 mg in immediate-release capsules (MNTX-IR) and
extended-release capsules (MNTX-ER) in 12 healthy subjects which responded to
loperamide with prolongation of the whole-gut transit time by >20.5 h
(>25% quartile of the sample).

| | | | | LOP + | LOP + | LOP + |
|---|---|---|---|---|---|---|
| | | Control | LOP | MNTX-SC | MNTX-IR | MNTX-ER |
| OCT | (h) | 3.92 ± 0.70 | 7.00 ± 3.55 | 6.42 ± 3.94 | 5.29 ± 2.78 | 5.67 ± 2.97* |
| WGT | (h) | 39.5 ± 8.56 | 77.3 ± 19.6 | 71.5 ± 17.2 | 71.3 ± 21.1 | 62.3 ± 16.1*/** |
| CTT | (h) | 35.6 ± 9.01 | 70.3 ± 19.4 | 65.1 ± 17.9 | 66.0 ± 21.6 | 56.7 ± 16.2*/** |

p < 0.05 Wilcoxon test,
*compared to LOP,
** compared to MNTX-IR

The study medication was safe and well tolerated. In the entire study, only headache (2×), constipation (1×) and meteorism (1×) were likely or definitely related to the study medication.

1. A method comprising administering a composition comprising an opioid receptor antagonist to a subject having severe constipation characterized by a whole gut transit time (WGT) of at least 65 h.

2. The method of claim 1, wherein the opioid receptor antagonist is a μ-receptor antagonist.

3. The method of claim 2, wherein the μ-receptor antagonist is comprising naloxone, naloxonesulfate, methylnaloxone, naltrexone, or a pharmaceutically acceptable salt thereof.

4. The method of claim 1, wherein the severe constipation is not an opioid-induced constipation.

5. The method of claim 1, wherein the whole gut transit time is determined using radio-opaque markers.

6. The method of claim 1, wherein the patient is characterized by a whole gut transit time of at least 65 to 100 h.

7. The method of claim 1, wherein the composition releases the opioid receptor antagonist independently of the ambient pH of the gastrointestinal tract.

8. The method of claim 1, wherein the composition is designed as a once-a-day formulation.

9. The method of claim 1, wherein the composition is designed as a twice-a-day formulation.

10. The method of claim 1, wherein the WGT comprises a colon transit time of 55 to 90h.

11. The method of claim 1, wherein the WGT is 65 to 180 h.

12. The method of claim 1, wherein the composition further comprises a matrix.

13. The method of claim 1, wherein the composition is formulated as a capsule.

14. The method of claim 1, wherein the opioid receptor antagonist is not naloxone.

15. The method of claim 1, wherein the composition further comprises an opioid.

16. The method of claim 1, wherein the severe constipation is an opioid-induced constipation.

17. The method of claim 16, wherein the opioid-induced constipation is not caused by oxycodone.
